(19) United States
(12) Patent Application Publication
(10) Pub. No.: US 2003/0198694 A1
(43) Pub. Date: Oct. 23, 2003

(54) Preparation antioxidants enriched functional food products from sugar cane and beet

(76) Inventor: Chung Chi Chou, South Huntington, NY (US)

Correspondence Address: Chung Chi Chou, 103A Pidgeon Hill Rd., South Huntington, NY, 11746, US

(21) Appl. No.: 10/127,141

(22) Filed: Apr. 22, 2002

(51) Int. Cl. A61K 35/78; A23B 4/00; A23D 7/00

(52) U.S. Cl. 424/725; 426/541

(57) ABSTRACT

Functional food products with excellent antioxidative strength have been prepared from natural sugar cane and beet. The processes used include one or more of the following: Clarification, Crystallization, Chromatographic separation process, Adsorption on/Desorption from adsorbents, Ion exchange resin decolorization and regeneration, and Ultra-Nano membrane filtration. The antioxidative capacities of the products are quantified in term of ORAC (Oxygen Radical Absorbance Capacity) unit as per analytical method developed at the Agricultural Research Services of the U.S. Department of agriculture.

### REFERENCES TO RELATED APPLICATIONS

(a) U.S. Patents

| Patent No. | Date | Authors | |
|---|---|---|---|
| 5,179,012 | Jan. 12, 1993 | Gudin,et al | 435/125 |
| 5,017,397 | May 21, 1991 | Nguyen,et al | 426/542 |
| 4,232,122 | Nov. 4, 1980 | Zilliken | 435/52 |
| 4,218,489 | Aug. 19, 1980 | Zilliken | 426/545 |

(b) Other References Cited

(1) Farber, L. and Carpenter, F. G., Plant pigments as colorants in cane sugar, proceeding 1972 Tech. Sess. Cane Sugar Refining Research, p. 23

(2) Mary An Godshall and Earl J. Roberts, phenolics in sugar products, proceeding of the 1982 sugar processing conference, P. 47

(3) Margaret A. Clark, W. S. C. Tsang and M. A. Godshall, structure of colorants, proceeding of the 1988 sugar processing research conference, 1988, P.183

(4) Richard Riffer, non-sugar and sugar refining, chapter 36, Handbook of Sugar Refining (2000), edited by Chung Chi Chou, published by John Wily & Sons, Inc. New York.

(5) Judy McBride, Can Foods Forestall Aging, February, 1999 issue of Agricultural Research Magazine, USDA

(6) L. Farber and F. Carpenter, Proc. Tech. Sess., Cane sugar Refining. Res., Boston, 1970

(7) Donald E. Pszczola, Anti0oxidants from preserving food quality to quality of life, vol. 55, no. 6. June 2001, Food Technology

(8) Susanne J. Klahorst, Abstract on Anti-oxidants, May 2001, Food Product Design

(9) Judy McBride, High-ORAC foods may slow aging, February 1999 issue of Agricultural Research Magazine, USDA

(10) Yukie Nagai, Takco Mizutani, Hiroshi Iwabe, Saiichi Araki, and Mamoru Suzuki Physiological function of sugar cane extracts. Technical proceeding of Sugar Technologists, Inc. 2001.

(11) Chung Chi Chou and A. E. Rizzuto, Acidic nature of sugar colorants, proceedings of the 1972 technical Session on cane sugar refining research, Agricultural Research Service, USDA

(12) Frank G. Carpenter, chapter 17, decolorization, Cane sugar handbook 11^{th} edition by James C. P. Chen, published by John Wiley and sons, 1985.

(13) James C. P. Chen and Chung Chi Chou, Cane sugar handbook 12^{th} edition, Chapter 5 & 12, published by John Wiley and Sons, 1993

(14) Mumir Chervan, Ultrafiltration Handbook, Technomic Publishing Company, Inc. Lancaster, Pa., 1986,.

### BACKGROUND OF THE INVENTION

Antioxidants have been reported to have beneficial effect as stabilizers for food and potentially useful in prevention and/or treatment of some diseases. Various attempts have been made to produce antioxidants: (a) Zilliken has patented methods to produce antioxidants (U.S. Pat. Nos. 4,218,489 and 4,232,122) from fermented soybean product. However, the process involved extraction using petroleum based solvents which are difficult to operate/handle and render product quality problem related to the use of solvents, (b) Nguyen, et al patented a process (U.S. Pat. No. 5,017,397) for extracting antioxidants from Labiatae herbs. The process has limited practical applications because of the use of supercritical fluid extraction and fractionation with carbon dioxide. The process would be expensive both in capital and in operating costs, and (c) Gudin, et al patented a process (U.S. Pat. No. 5,179,012) for production of antioxidants from a microorganism culture in a photobioreactor by photosynthesis. The process involves complex operations and is subject to strict process control to make desired products. All the above processes also have limitations in producing large quantity of products.

This invention has the following advantages over the above mentioned existing arts: (a) the processes use well established unit operations/technologies with innovative modifications, (b) the sources of raw material are sugar cane and beet. Since sugar cane is known to be the most productive plant in production of carbohydrate per unit of farm land, the supply of raw material for antioxidants production is unlimited and inexpensive, and most importantly, (c) the antioxidants are from natural plant extracts-sugar cane and beets.

It has been well documented that sugar cane and beet plants derived compounds include flavonoids, substituted phenolics and polyphenolics. Farber and Carpenter (1) reviewed the literature on the subject of phenolics in sugar cane till 1972. Godshall and Roberts summarized the role of phenolics in sugar product in relation to the nature of colorants (2). The structure of colorants which partially derived from phenolic based plants pigments was discussed by Margaret A. Clark (3). More recently, Richard Riffer (4) described phenolics as a small but important part of non-sugar in the sugar processing of raw sugar and reported four flavonals and 25 flavones had been identified in sugar cane. A total of over 4000 flavonoids was reported to constitute a major dietary antioxidants considered to be responsible for a large part of antioxidative power of fruits and vegetables as reported by Judy McBride of Agricultural Research Service (5). In addition, a number of naturally occurring pigments in sugar cane, such as chlorogenic acid, hydroxy cinnamic acid, were identified by Farber (6). These compounds were reported to be very effective in antioxidative power (7, 8). Although these compounds are well known phytochemicals widely distributed in plants, including sugar cane and beets, and extensively studied by researchers in the sugar industry, however to-date, no attempt has ever been made by sugar researchers to correlate these findings to anti-oxidative activities as related to health. All the studies have focused on the relationship between these substances and color in the sugar juice/sugar liquor, and the mechanism of their removal as part of colorants in sugar refining/manufacturing process to make white/refined sugar. This inventor is the first, to the best of my knowledge, to discover the excellent beneficial antioxidative capabilities of antioxidants from sugar cane and beets, and methods to produce it.

Food rich in antioxidants, as measured in ORAC unit, may protect cells and their components from oxidative damage based on studies of animals and human blood at the Agricultural Research Service's Human Nutrition Research Center on Aging at Tuft University in Boston (9). ARS is the chief scientific agency of U.S. Department of Agriculture. ORAC, the abbreviation of Oxygen Radical Absorbance Capacity, is a laboratory analytical method for determination of total antioxidative function of food and other substances. The method is developed by USDA scientists Drs. Guohum Cao and Donald L. Prior. Intake of high ORAC foods may help to reduce risk of diseases associated with aging of both body and brain. Cao and Prior suggested that daily intake of 3000 to 5000 ORAC units should have significant impact on plasma and tissue anti-oxidative capacity. The ORAC values of top-scoring fruit and vegetable, prunes and kale, were reported to be 5770 and 1770 per 100 grams respectively (9)

In literature search covering all field only one paper, published in August 2001 (10) by a Japanese company, describes the physiological function of sugar cane extracts. In this study, four extracts were obtained using chromatographic separation process, ion exchange resin process and hot water extraction of cane bagasse respectively. Certain extracts were found to exhibit phylotic effect, vaccine adjuvant effect and protection effects on liver injuries on studies using rat. Two extracts were shown to have super-oxide anion scavenging activities (SOD), a measure of antioxidative capacity according to the authors. However, the authors concluded that the relationship between anti-oxidative capacity of the extracts and other physiological function is not clear, as is the mechanism of such effect. It is unknown the correlation between SOD activity and ORAC unit.

In this patent application, the inventor describes methods to separate, enrich & concentrate antioxidants from sugar products to prepare high-ORAC functional food products for human consumption.

### SUMMARY OF THE INVENTION

Sugar cane and beet embody highly color substances containing polyphenolics, flavonoids and other compounds with significant anti-oxidative capacities. The beneficial health effect of plants' antioxidants has been widely reported in the literature. However, no patent reference is available citing sugar cane/beets as the sources for productions of antioxidants as functional food products. This inventor is the first to study and develop processes to produce functional food products with exceptional antioxidative capabilities from sugar cane and beets. The antioxidative power is quantified in term of ORAC unit, the abbreviation of Oxygen Radical Absorbance Capacity, a laboratory analytical method developed by USDA scientists for determination of total antioxidative function of food and other substances.

The invention covers the preparation of high ORAC, antioxidants enriched functional food products from sugar cane and beets employing a single or combination of standard chemical engineering separation processes, with modifications when needed: clarification, evaporation, crystallization, chromatographic techniques, adsorption/desorption, ion exchange decolorization and regeneration, and membrane Ultra- and Nano-filtrations.

Any and/or combination of the above processes can be used to produce antioxidants enriched functional food products from aqueous sugar containing solution from sugar cane and beets.

### DESCRIPTION OF THE DRAWING

There are FIG. 1 and FIG. 2 in one drawing.

FIG. 1 is a simplified flow diagram for raw sugar and plantation white sugar production.

FIG. 2 is a simplified flow diagram for production of refined sugar. The drawing depicts processes for sugar production. The same principle of each process is used as part of the processes for production of high OARC, antioxidants enriched products.

### DESCRIPTIONS OF THE INVENTION

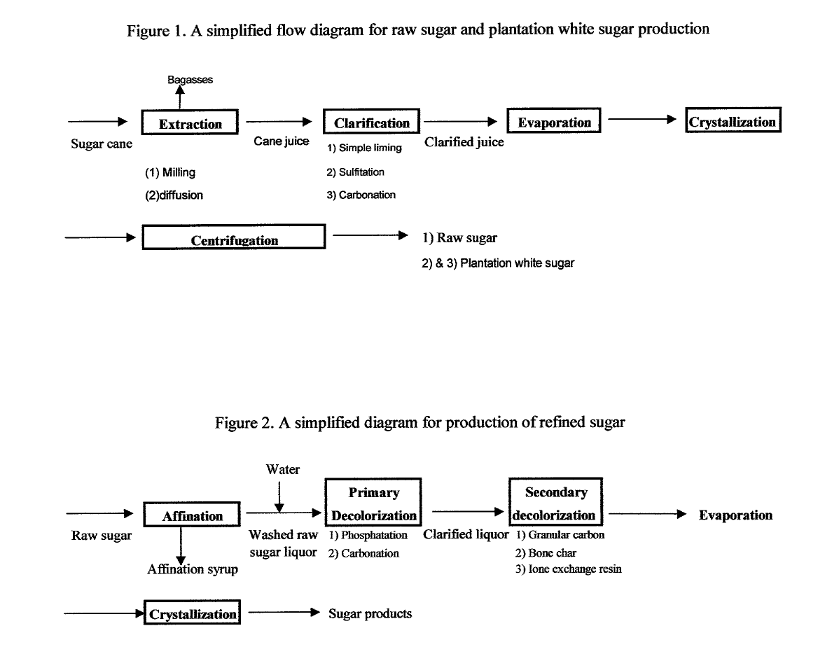

To illustrate preparation of high ORAC antioxidants enriched products, an understanding of sugar manufacturing processes is essential as shown in FIGS. 1 & 2. These standard processes, such as clarifications, decolorization/adsorption, ion exchange process, crystallization can be found in textbooks in great details (12,13). FIG. 1 shows a simplified flow diagram for raw sugar and plantation white sugar production. FIG. 2 shows a simplified flow diagram for production of “refined sugar”.

(A) Clarification: As shown in FIG. 1, sugar juice is extracted from sugar cane or beet either by milling or diffusion after initial crushing and/or shredding. The sugar juice normally has a color of between 5000 ICU (international color unit) to 25,000 ICU, which consists of about 78 to 90% sucrose and the balance of non-sucrose on dried basis. The non-sucrose fraction includes ash, polysaccharide, gum, waxes, colorants, polyphenolics, flavonoids and other antioxidants etc. The sugar juice at about 15 brix (% dry solid) is then clarified, generally by three different processes. To make raw sugar with color ranging from 700 to 8000 ICU, simple Timing clarification is used.

To make plantation white sugar with color ranging from 80 to 250 ICU, either sulfitation or carbonation process can be used. Raw sugar is subject to further refining process to make white sugar with color ranging from 10 to 65 ICU. Plantation white sugar is for direct consumption, generally in developing countries. Simple liming clarification removes the least non-sucrose, including color and other organic matters, among three processes. In general all three clarification processes are followed a filtration step as needed in order to meet requirements as food grade products, Beet juice is clarified by carbonation. The sulfitation processes generally include first sulfitation and second sulfitation, and reduce up to 40% of juice color. The carbonation process normally is to be followed by another simple sulfitation and remove up to 65% color. Since color is a degree of measurement of antioxidants, processes with high color removal efficient, such as carbonation would result in clarified juice with less antioxidants constituents.

All the food grade products for human consumption need to be manufactured in accordance to regulatory requirements with respect to GMP (god manufacturing practice), use of direct and indirect additives, and processing aids, etc. Therefore, raw sugar juice, which is full of suspended solids and microbes, need to be clarified first before further processing by evaporation, crystallization and centrifugation. The processes most used are simple liming, sulfitation, phosphatation and carbonation. As discussed earlier, certain clarification process, such as carbonation, absorbs/removes significant quantity of colorants/antioxidants from sugar stream and disposed off as carbonate cake. For example, the total phenolics contents of a cane mixed juice is 1127 ppm, the carbonated clarified juice has a content of 298 ppm, a 73.5% removal rate. Another sample with initial phenolics contents of 1,966 ppm, it dropped to 280 ppm, an 85.8% reduction after carbonation. Therefore, appropriate processes must be developed to clarify raw juice without significant removal of high ORAC constituents, such as polyphonolics, flavonolds, etc. We found that, clarification by simple liming and/or soda ash preserved/retained high ORAC constituents in the clarified juice as shown below:

The details of clarification of raw juice or sugar liquor are described in several textbooks, such as Cane Sugar Handbook (13). In general raw juice/sugar liquor at temperature of 50° C. to 80° C. is coarse screened to remove large suspended particles, followed by addition of about 100 ppm to 1% of processing aids and reheated to between 85° C. to 110° C. before entering a clarifier. The retention time in clarifier range from 30 minutes to 3 hours. The time, temperature and amount of processing aids depend on the purity of sugar solution being treated. Since sugar juices purity usually varies from 78 to 90% depending on weather, crop seasons and farm region, the important criteria is to select conditions which would produce clear clarified sugar solution without removing significant amount of high ORAC anti-oxidants. We have found through out the tests that processing aids dosage of less than 1% meet the requirements.

Example: We have found that a coarse screened raw syrup has a very high ORAC value of 35,600 unit/100 gram of dry solid. Another sample produced an ORAC value of 27,226 units/100 gram. The inventor was pleasantly shocked to find such a high ORAC unit for the cane juice. Previous findings for “B” and “C” molasses from a carbonation factory only gave 5,755 and 4,835 ORAC units per 100 gram on dried basis. However, these products are not food grade because the sugar solution is not clarified. For comparison purposes, it should be noted again that the ORAC value of prunes, oranges, kale and spinach are 5,770, 750, 1,770 and 1,260 per 100 gram of sample as received. If these units were converted to dry solid basis, the value would be much higher for these fruits and vegetables. These data are published with copy right by Agricultural Research Service in USDA Agricultural Research Magazine on February 1999 issue.

Example: (a) A sample of cane syrup clarified to meet food grade requirements, by lime addition as processing aid produced an ORAC value of 36,051 unit/100 gram dried solid. (b) Another sample of cane syrup clarified by lime addition had an ORAC value of 29,830 unit. (c) A sample of cane juice clarified with soda ash produced an ORAC value of 36,491 units per 100 grams of dried solid. (d) Another soda ash treated sample has an ORAC value of 25,228 units. With all the processing aids used for clarification, such as liming, soda ash addition, carbonation, sulfitation, and phosphatation, the carbonation with large quantity of lime followed by gassing with carbon dioxide for pH control, removes the most color/antioxidants. Therefore, conventional carbonation is not suitable for preparation of high ORAC product. For example, (e) a carbonated syrup only gave 4,835 units of ORAC even after concentration twice by crystallization.

Treatment of sugar containing solution using chemical processing aids, such as lime, sulfur dioxide, soda ash or phosphoric acid, removes macromolecules and suspended solid, including microbes without significant removal of antioxidants.

The phosphation and carbonation in the second step of sugar refining remove approximately 55 to 60% colorant and therefore the antioxidants. Since the resulting carbonate cake or phosphate scum is subsequently discarded/disposed of. It is very difficult, at least economically, to recover antioxidants from those waste streams.

(B) Crystallization: Referring back to FIG. 1, the clarified juice is further subject to crystallization in vacuum pans after evaporation. The massecuite from crystallization in a vacuum pan is then centrifuged to separate mother liquor from crystal sugar. Since crystallization is one of the best purification steps, with about 50% yield of sucrose, the colorants/anti-oxidants normally remained in the mother liquor. Therefore, crystallization is an excellent way to enrich/concentrate antioxidants for production of high-ORAC functional food products.

Referring to FIG. 2, for refining of raw sugar to make refined sugar, the first step is affination, which involves mechanically “washing” the raw sugar with recycled affination syrup. The affination process mechanically removes about 75 to 85% of total non-sucrose, including colorants/antioxidants, from the surface of raw sugar crystal, indicating exclusion of non-sugar during the crystallization. This again indicates the effectiveness of crystallization step as an excellent way for concentration of anti-oxidants into the mother liquor.

Example: Crystallization of “A” syrup with ORAC unit of 4,046 gave a B molasses with enriched ORAC of 6,604 and a sugar depleted with ORAC at 1867 unit.

(C) Chromatographic separation process: The process is widely used in the beet industries to recover additional sucrose from molasses. It basically separates the molasses into two fractions: sucrose fraction with about 90% recovery and a second fraction of non-sucrose stream, which include organic and inorganic constituents. In case of cane molasses a small third fraction of invert sugars is also obtained. In practice any process stream in a sugar plant can be subjected to chromatographic fractionation to obtain a non-sugar fraction. It is well accepted that, the concentration factors for non-sugar fractions from a conventional chromatographic separation process are six and two respectively for cane juice and molasses.

Example: A “C” molasses with ORAC of 5,755 would give a nonsugar fraction with ORAC value of 11,510 units.

(D) Adsorption/Desorption: The secondary decolorization step in FIG. 2 involved the use of adsorbents, such as granular carbon and/or bone char. These processes remove, by adsorption onto their surface, over 80% of colorants/antioxidants from sugar containing solution. In sugar plants, these exhausted granular carbon and bone char are thermally regenerated/reactivated by burning off adsorbed colorants and other organic matter under limited oxygen atmosphere at about 1800° F. and 1100° F. respectively. We have developed an economical way to desorb or to strip off the colorants/antioxidants from these adsorbents using alkaline solution, to give concentrated high-ORAC, antioxidants enriched products.

Since many colorants, including polyphenolics and flavonoids, posses aromatic character, they are easily adsorbed onto hydrophobic carbon surface. After the “decolorization” or adsorption of color onto its surface, the carbon can be washed with water and then the remaining colorants/antioxidants can be desorbed, eluted, or strip off the carbon surface using 0.5 to 2% sodium hydroxide solution. This adsorption/desorption phenomenon was described in some detail by Chou and Rizzato (11). Although Amberlite XAD-2 (made by Rohm and Haas Company) were used in their study, the adsorbent is known to have similar hydrophobic nature as carbon and follow the general theory of adsorption (12). Adsorbents such as XAD-2 and XAD-1150 (Rohm and Haas) have minimal functional groups for ion exchange, but have excellent adsorption capacities through their hydrophobic surface similar to carbon.

We have discovered that the use of carbon and other similar adsorbents, such as Amerlite XAD-2, XAD-1150 via adsorption/desorption process described above is exceptionally effective for preparation of concentrated antioxidants mixtures from aqueous sugar containing solution. For further purification and concentration of these antioxidants mixtures contained in the eluents from the desorption process, or desorbed/stripped off solutions, strong acid cation (SAC) exchange resin in hydrogen form (H⁺form), such as Tulsion T-42MP H⁺, is used to remove the ash (deashing) including NaOH used for elutions, from the eluents or desorbed/stripped off solutions.

Example: (1) XAD adsorbents column test: (a), twenty liters of clarified cane syrup, with an initial ORAC value of 54,172 unit per 100 gram dried solid, at 60 brix and 65° C. was pumped through a 2.5×60 cm column filled with Rohm and Hass XAD-1150 as adsorbent, (b) the column was then wash/desweetened off with deionized hot water, (c) the water washed column was then eluted/washed with 1 to 2% NaOH solution, (d) the antioxidants containing effluents (eluents/desorbed solutions) from above step (c) is then passed through another column filled with deashing strong acid cation resin (SAC), TulsionT-42MP H⁺form, to remove ash including NaOH in the eluents, (e) the eluents from above step (d) was concentrated to give a functional food products containing exceptionally high antioxidants with final ORAC value of 1.26 millions per 100 gram on dried solid basis.

It should be noted from this test that there was a 23.2 folds (times) increase in the antioxidants concentration produced by this adsorption/desorption process.

(2) Granular activated carbon (GAC) column test: (a), twenty liters of clarified cane syrup, with an initial ORAC value of 54,172 unit per 100 gram dried solid, at 60 brix and 65° C. was pumped through a 2.5×60 cm column filled with granular activated carbon (GAC), (b) the column was then washed/desweetened off with deionized hot water, (c) the water washed column was then eluted/washed with 1 to 2% NaOH solution, (d) the antioxidants containing effluents (eluents/desorbed/stripped off solutions) from above step (c) is then passed through another column filled with deashing strong acid cation (SAC) resin, TulsionT-42MP H⁺form, to remove ash including NaOH in the eluents, (e) the eluents from above step (d) was concentrated to give a functional food products containing high antioxidants with ORAC value of 64,230 unit per 100 gram on dried solid basis. The adsorption/desorption process using granular carbon adsorbents still produced significantly higher antioxidants product

(3) Granular activated carbon (GAC) batch test. (a) A 30 brix “C” molasses with an initial ORAC of 5,755 unit is mixed with granulated activated carbon (GAC) at 80° C. for two hour. (b) After filtering out the sugar solution, the carbon is first washed/desweetened with hot water, (c) the washed carbon was then mixed with sodium hydroxide solution for two hour at pH 9 to desorb/strip off antioxidants from carbon surface and then filtered. The filtrate has an enriched ORAC of 18,036 unit on dried basis, at the same purity of 50% as that of “C” molasses.

(4) A repeated test of above (3) gave a product with an ORAC unit of 18,436 as compared to 18,036 ORAC unit of test (3).

(E) Ion Exchange Resin decolorization and regeneration. In sugar processing, ion exchange resin, exhausted with color exchange capacity, is reactivated/regenerated with about 8% sodium chloride and 0.5% caustic soda brine solution (regenerant). About 90% of colorants exchanged on to the resin is desorbed and concentrated in the brine regenerants. This regenerant would be a good source of antioxidants if a nano-membrane process or strong acid cation (SAC) resin is used to separate/remove sodium chloride, caustic soda and other ash from colorants/antioxidants.

Example: A 60 purity “B” molasses with an initial ORAC value of 4,186 was passed through ion exchange resin at 65 brix and 80° C. After the resin was exhausted with colorants/antioxidants, the resin was washed with hot water and then regenerated by elution with caustic brine solution to desorb and strip off colorants/antioxidants. The brine solution containing antioxidants (regenerant) has an ORAC value of 16,744 at the same 60% purity of “B” molasses, a four time increase in antioxidants concentration. The antioxidants mixture can further be purified/concentrated by nano membrane filtration or by strong acid cation (SAC) deashing resin as discussed before.

(F) Cross flow tangential membrane Ultra- and Nano-filtration process is another good way to produce high ORAC food products from sugar processing stream. Cross flow tangential membrane filtration is widely used in the corn industries for specialty products manufacturing. The theory and practices of the processes can be found in the Ultrafiltration Handbook by Munir Chervan (14). Membrane filtration, by definition, is a process to separate two or more components from a fluid stream. The degree of separation will depend on the particle or molecular size (or molecular weight) of the components and the pore size of the membrane. Many vendors supply a series of membranes with various molecular weight cut-off limits. For example, Koch membrane system K-131 has a molecular weight (MW) cut-off limit of MW=10,000. Most antioxidants with molecular weight larger than 10,000 will be retained and concentrated on the retentate side. Sucrose (MW=342), glucose, fructose, water and inorganic ash will pass through the membrane as permeates stream. K-328, MPF-36 and MPF-34 membranes have molecular weight cut-off limits of 5000, 1000 and 200 respectively. You can select the type of membranes to achieve your separation objectives. Strength of antioxidants in the retentate can also be controlled by the concentration factor of the membrane separation process. Concentration factor of 1X represent 50% recovery, concentration factor of 10X represent 90% recovery.

Example: A “B” molasses with an initial ORAC value of 6,604 unit was diluted to 10 brix and passed through UF membrane with a molecular weight cut off limit of 50,000 to 100,000. The test gave an antioxidants enriched retantate with an ORAC of 6,651 at one (1) X concentration factor and 12,015 at concentration factor of nine (9) X. Another test gave a retantate with ORAC value of 8,807 unit at a concentration factor of nine (9) X. Although there were some increase of ORAC value for the retantate at a concentration factor of 1 X in these tests, It is obvious that a membrane with less than 50,000 molecular weight cut off limits will be needed to be more effective in concentrating antioxidants.

I claim:

1) Methods for the manufacturing of antioxidants enriched antioxidative functional foods from aqueous sugar containing solution, extracted from sugar cane or sugar beet, containing sugar, organic and inorganic non sugar, comprising clarification with processing aid(s) and/or one or more of the following processes: crystallization/recrystallization, chromatographic separation process, adsorption and desorption using adsorbents, regeneration from ion exchange decolorization resin, cross flow tangential ultra membrane filtration and nano membrane filtration, to enrich, purify, and concentrate high antioxidants functional foods.

2) A process according to claim (1), characterized in that the above said aqueous sugar solution is clarified, using one or more of the following processing aids: lime, soda ash, sulfur dioxide, aluminum chloride and carbon dioxide to produce clarified sugar containing solution rich in antioxidants as functional food products.

3) A process according to claim (2), characterized in that the clarified sugar containing solution is followed by evaporation and crystallization processes to give antioxidants enriched functional food products, either in diluted or in concentrated or in dried form, and crystal sugar depleted with antioxidants.

4) A process according to claim (2), characterized in that the clarified sugar containing solution is subject to a chromatographic process to give antioxidants enriched functional food products, either in diluted or in concentrated or in dried form.

5) A process according to claim (4), characterized in that the high antioxidants enriched liquid products are further subject to ion exchange deashing resin or nano membrane filtration, to remove ash components to give low ash high antioxidants enriched food products, either in diluted or in concentrated or in dried form.

6) A process according to claim (2), characterized in that the clarified sugar containing solution is subject to an adsorption process by passing through, or in contact with adsorbents, such as granular or powdered carbon, bone char and other adsorbents, such as Rohm and Hass XAD-series products.
The antioxidants adsorbed/retained on the adsorbents via the said adsorption process are extracted or eluted from/stripped off with alkaline solution, such as caustic soda, soda ash solution to give a high antioxidants enriched functional food products, either in diluted or in concentrated or in dried form.

7) A process according to claim (6), characterized in that the high antioxidants extracts/eluents from adsorbents is further subject to ion exchange deashing resin or nano-membrane filtration to remove ash components to give low ash high antioxidants enriched food products, either in diluted or in concentrated or in dried form.

8) A process according to claim (2), characterized in that the clarified sugar containing solution is subject to ion exchange processes by passing through or in contact with ion exchange resins, followed by regeneration or elution of adsorbed and/or exchanged antioxidants from the ion exchange resins using an alkaline brine solution containing about 8% sodium chloride and about 1% sodium hydroxide, to give high antioxidants enriched functional food products, either in diluted or in concentrated or in dried form.

9) A process according to (8), characterized in that the high antioxidants regenerant/eluents are further subject to ion exchange deashing resin or nano-membrane filtration to remove ash components give low ash high antioxidants enriched food products, either in diluted or in concentrated or in dried form.

10) A process according to claim (2), characterized in that the clarified sugar containing solution is subject to ultra- or nano-membrane filtration with maximum pore size equivalent to molecular cut-off limit of 75,000 to give a high antioxidants enriched food retentate product, and a food permeate product, either in diluted or in concentrated or in dried forms.
